(19) United States
(12) Patent Application Publication
(10) Pub. No.: US 2018/0251425 A1
(43) Pub. Date: Sep. 6, 2018

(54) 2,3,5 TRISUBSTITUTED PYRROLE DERIVATIVES AS TOPOISOMERASE INHIBITORS AND THERAPEUTIC USES THEREOF

(71) Applicant: UNIVERSITY OF MYSORE, Mysuru (IN)

(72) Inventors: Ananda Hanumappa, Mysuru (IN); Sharath Kumar Kothanahally S, Mysuru (IN); Mahesh Hegde, Mysuru (IN); Sathees C. Raghavan, Mysuru (IN); Kanchugarakoppal S Rangappa, Mysuru (IN)

(73) Assignee: UNIVERSITY OF MYSORE, Mysuru (IN)

(21) Appl. No.: 15/753,888

(22) PCT Filed: Feb. 26, 2016
(86) PCT No.: PCT/IN2016/050069

§ 371 Date: Feb. 20, 2018

(30) Foreign Application Priority Data
Aug. 17, 2015 (IN) 4307/CHE/2015

(51) Int. Cl. C07D 207/34; C07D 405/04; C07D 401/04; C07D 409/14; C07D 409/04; A61P 35/00

(52) U.S. Cl. CPC C07D 207/34; C07D 405/04; A61P 35/00; C07D 409/14; C07D 409/04; C07D 401/04

(57) ABSTRACT

The compounds of Formula (1) having topoisomerase inhibitory effect includes

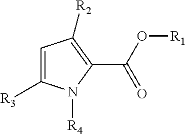

wherein, R1 is selected from a group consisting of H, OR5, optionally substituted C1-C12 alkyl, haloalkyl, C2-C12alkenyl, C2-C12alkynyl, C1-C12alkyloxy, C1-C12haloalkyloxy, C2-C10 heteroalkyl, C3-C12 cycloalkyl, C3-C12cycloalkenyl, C2-C12heterocycloalkyl, C2-C2 heterocycloalkenyl, C6-C18aryl, and C1-C18heteroaryl;
R2, R3 and R4 are independently selected from a group consisting of H, halogen, CN, —NO2, SH, CF3, OH, CO2H, CONH2, OCF3, optionally substituted C1-C12alkyl, optionally substituted C1-C12haloalkyl optionally substituted C2-C12alkenyl, optionally substituted C2-C12alkynyl, optionally substituted C1-C12alkyloxy, optionally substituted C1-C12haloalkyloxy, optionally substituted C2-C12heteroalkyl, optionally substituted C3-C12cycloalkyl, optionally substituted C3-C12 cycloalkenyl, optionally substituted C2-C12 heterocycloalkyl, optionally substituted C2-C12 heterocycloalkenyl, optionally substituted C6-C18aryl, and optionally substituted C1-C18heteroaryl;
R5 is selected H, optionally substituted C1-C12alkyl, optionally substituted C2-C12alkenyl, optionally substituted optionally substituted C1-C12 haloalkyl, optionally substituted C3-C12cycloalkyl, optionally substituted C6-C18aryl, and optionally substituted C1-Ci18heteroaryl;
or a pharmaceutically acceptable salt, N-oxide, or prodrug thereof.

### CROSS REFERENCE TO RELATED APPLICATIONS

The present application is a national phase application of international application number PCT/IN2016/050069, filed on Feb. 26, 2016 which claims priority from, IN Application Number 4307/CHE/2015, filed on Aug. 17, 2015, the disclosure of which is hereby incorporated by reference herein.

### FIELD OF INVENTION

The present invention relates to inhibitors of topoisomerase activity. The present invention specifically relates to pyrrole derivatives and their use as topoisomerase inhibitors and their therapeutic uses thereof. The invention further relates to use of pyrrole derivatives used for treating cell-proliferative disorders. In some instances, these compounds have anticancer activity.

### BACKGROUND OF INVENTION

Globally, one of the major challenges that the health system faces today is to provide an effective treatment for cancer, which significantly contributes to the global disease burden. With present methods of treatment, one-third of patients are cured with local measures, such as surgery or radiation therapy, which are quite effective when the tumor has not metastasized by the time of treatment. Earlier diagnosis might lead to increased cure of patients undergoing such local treatments. However, in many cases, early micrometastasis is a characteristic feature of the neoplasm, indicating that a systemic approach such as chemotherapy may be required, often along with a local treatment method, for effective cancer management.

Cancer chemotherapy can be curative in certain disseminated neoplasms that have undergone either gross or microscopic spread by the time of diagnosis. These include testicular cancer, diffuse large cell lymphoma, Hodgkin's disease and choriocarcinoma as well as childhood tumors such as acute lymphoblastic leukemia. For other forms of disseminated cancer, chemotherapy provides a palliative rather than curative therapy. Effective palliative therapy results in temporary clearing of the symptoms and signs of cancer and prolongation of useful life. Advances in cancer chemotherapy have recently provided evidence that chemical control of neoplasia is possible for a number of cancers.

One category of drugs used for cancer therapy is topoisomerase inhibitors. Topoisomerases are vital nuclear enzymes which function to resolve topological dilemmas in DNA, such as overwinding, underwinding and catenation, which normally arise during replication, transcription and perhaps other DNA processes. These enzymes allow DNA to relax by forming enzyme-bridged strand breaks that act as transient gates or pivotal points for the passage of other DNA strands. Topoisomerase-targeting drugs appear to interfere with this breakage-reunion reaction of DNA topoisomerases. In the presence of topoisomerase-active agents, an aborted reaction intermediate, termed a ‘cleavable complex’, accumulates and results in replication/transcription arrest, which ultimately leads to cell death. These compounds inhibit the action of topoisomerase enzymes which play a role in the replication, repair, genetic recombination and transcription of DNA. An example of a topoisomerase inhibitor is camptothecin, a natural compound that interferes with the activity of topoisomerase I, an enzyme involved in DNA replication and RNA transcription. Camptothecin and the camptothecin analogues topotecan and irinotecan are approved for clinical use.

Camptothecin and its analogues are effective in cancer chemotherapy by interfering with the breakage/reunion actions of topoisomerase I. The compounds stabilize and form a reversible enzyme-camptothecin-DNA ternary complex which prevents the reunion step of the breakage/union cycle of the topoisomerase reaction.

One problem with camptothecin is its water insolubility, which hinders the delivery of the drug. Another problem with camptothecin and its analogues is that the compounds are susceptible in aqueous environments to hydrolysis at the α-hydroxy lactone ring. The lactone ring opens to the carboxylate form of the drug, a form that exhibits little activity against topoisomerase I.

Hence, a lot of research has been focused towards finding an inhibitor of topoisomerase-II activity. One such example of a topoisomerase-II inhibitor is Doxorubicin which inhibits the progression of the enzyme topoisomerase II, which relaxes supercoils in DNA for transcription. Doxorubicin stabilizes the topoisomerase II complex after it has broken the DNA chain for replication, preventing the DNA double helix from being resealed and thereby stopping the process of replication. Although these are desirable effects in cancer treatment, Doxorubicin has shown to have adverse effects causing life threatening heart damage. Although there is extensive research being carried out to overcome these adverse effects of existing treatment options, there is however, still a need in the art for effective topoisomerase-II inhibitors and the inventors of this invention have attempted to address such need.

### OBJECT OF THE INVENTION

The principal object of the invention is to provide compounds that are inhibitors of topoisomerase activity.

A further object is to provide a pharmaceutical composition containing a compound that is an inhibitor of topoisomerase activity and a pharmaceutically acceptable excipient, diluent or carrier.

A further object is to provide a method of prevention or treatment of cancer by inhibiting cell proliferation using inhibitors of topoisomerase.

### BRIEF DESCRIPTION OF FIGURES

FIG. 1 describes the Evaluation of antiproliferation studies following addition of ASR6 on Molt4, Nalm6, Reh & K562 cells.

FIG. 2 describes the study of cell cycle progression of ASR6 on Molt4 cells at 24 h and 48 h time points.

FIG. 3 shows the measurement of loss of mitochondrial membrane potential followed by ASR6 treatment.

FIG. 4 describes the result of Annexin V-FITC staining of ASR6 treated Molt4 cells

FIG. 5 describes the effect of ASR6 on DNA relaxation catalyzed by Topoisomerase I

FIG. 6 describes the effect of ASR6 on DNA relaxation catalyzed by Topoisomerase II

### SUMMARY OF INVENTION

The present invention provides the compounds of Formula (1)

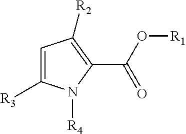

wherein, R1 is selected from a group consisting of H, OR5, optionally substituted C1-C12 alkyl, optionally substituted C1-C12 haloalkyl, optionally substituted C2-C12 alkenyl, optionally substituted C2-C12alkynyl, optionally substituted C1-C12 alkyloxy, optionally substituted C1-C12haloalkyloxy, optionally substituted C2-C10 heteroalkyl, optionally substituted C3-C12 cycloalkyl, optionally substituted C3-C12cycloalkenyl, optionally substituted C2-C12heterocycloalkyl, optionally substituted C2-C12 heterocycloalkenyl, optionally substituted C6-C18 aryl, and optionally substituted C1-C18heteroaryl;

R2, R3 and R4 are independently selected from a group consisting of H, halogen, CN, —NO2, SH, CF3, OH, CO2H, CONH2, OCF3, optionally substituted C1-C12alkyl, optionally substituted C1-C12 haloalkyl, optionally substituted C2-C12alkenyl, optionally substituted C2-C12 alkynyl, optionally substituted C1-C12alkyloxy, optionally substituted C1-C12haloalkyloxy, optionally substituted C2-C12 heteroalkyl, optionally substituted C3-C12cycloalkyl, optionally substituted C3-C12 cycloalkenyl, optionally substituted C2-C12 heterocycloalkyl, optionally substituted C2-C12 heterocycloalkenyl, optionally substituted C6-C18 aryl, and optionally substituted C1-C18heteroaryl;

R5 is selected H, optionally substituted C1-C12 alkyl, optionally substituted C2-C12alkenyl, optionally substituted optionally substituted C1-C12 haloalkyl, optionally substituted C3-C12cycloalkyl, optionally substituted C6-C18aryl, and optionally substituted C1-C18heteroaryl;

or a pharmaceutically acceptable salt, N-oxide, or prodrug thereof.

In some embodiments of the invention, R1 is selected from a group consisting of —CH3 and —CH2CH3.

In some embodiments of the invention, R2 is selected from a group consisting of optionally substituted phenyl, optionally substituted pyran, optionally substituted pyridine, optionally substituted naphthalene and optionally substituted thiophene.

In some embodiments of the invention R3 is selected from a group consisting of optionally substituted phenyl, optionally substituted pyran, optionally substituted pyridine, optionally substituted naphthalene and optionally substituted thiophene.

In some embodiments of the invention each optional substituent is independently selected from the group consisting of H, CH3, CH2CH3, CH2CH2CH3, CH(CH3)2, (CH2)3CH3, C1, Br, F, I, OH, NO2, NH2, CN, OCH3, OCH2CH2CH3, CF3, and OCF3.

In one preferred embodiment of the invention Formula (1) is a compound selected from a group consisting of:
Ethyl 3,5-diphenyl-1H-pyrrole-2-carboxylate;
Ethyl 5-(4-methoxyphenyl)-3-m-tolyl-1H-pyrrole-2-carboxylate;
Ethyl 3-(furan-2-yl)-5-(4-methoxyphenyl)-1H-pyrrole-2-carboxylate;
Ethyl 3-(3-nitrophenyl)-5-p-tolyl-1H-pyrrole-2-carboxylate;
Ethyl 3-(4-cyanophenyl)-5-p-tolyl-1H-pyrrole-2-carboxylate;
Ethyl 5-(4-chlorophenyl)-3-(pyridin-3-yl)-1H-pyrrole-2-carboxylate;
Ethyl 5-(4-chlorophenyl)-3-(naphthalen-2-yl)-1H-pyrrole-2-carboxylate;
Ethyl 3,5-di(thiophen-2-yl)-1H-pyrrole-2-carboxylate;
Ethyl 3-(4-bromophenyl)-5-(thiophen-2-yl)-1H-pyrrole-2-carboxylate;
Ethyl 5-(3,4-dimethoxyphenyl)-3-(4-(trifluoromethyl)phenyl)-1H-pyrrole-2-carboxylate;
Methyl 5-(3-methoxyphenyl)-3-(4-(trifluoromethyl)phenyl)-1H-pyrrole-2-carboxylate;
Ethyl 5-(3,4-dimethoxyphenyl)-3-(pyridin-4-yl)-1H-pyrrole-2-carboxylate;
Methyl 3-(3-nitrophenyl)-5-p-tolyl-1H-pyrrole-2-carboxylate;
Ethyl 3-(thiophen-2-yl)-5-(3,4,5-trimethoxyphenyl)-1H-pyrrole-2-carboxylate;
Ethyl 5-(3-methoxyphenyl)-3-(pyridin-4-yl)-1H-pyrrole-2-carboxylate.

In a further preferred embodiment of the invention, the compound with Formula (1) is Ethyl 5-(4-chlorophenyl)-3-(pyridin-3-yl)-1H-pyrrole-2-carboxylate

### DETAILED DESCRIPTION OF INVENTION

In this specification a number of terms are used which are well known to a skilled addressee. Nevertheless for the purposes of clarity a number of terms will be defined. As used herein, the term “unsubstituted” means that there is no substituent or that the only substituents are hydrogen.

The term “optionally substituted” as used throughout the specification denotes that the group may or may not be further substituted or fused (so asto form a condensed polycyclic system), with one or more non-hydrogen substituent groups. In certain embodiments the substituent groups are one or more groups independently selected from the group consisting of halogen, ═O, ═S, —CN, —NO2, —CF3, —OCF3, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxy alkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyi, acylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)Ra, —C(═O)ORa, C(═O)NRaRb, C(═NOH)Ra, C(═NRa)NRbRc, NRaRb, NRaC(═O)Rb, NRaC(═O)ORb, NRaC(═O)NRbRc, NRaC(═NRb)NRcRd, NRaSO2Rb, —SRa, SO2NRaRb, —ORa, OC(═O)NRaRb, OC(═O)Ra and acyl, wherein Ra, Rb, Rc and Rd are each independently selected from the group consisting of H, optionally substituted C1-C12 alkyl, optionally substituted optionally substituted C2-C12alkenyl, optionally substituted C2-C12alkynyl, optionally substituted C2-C10 heteroalkyl, optionally substituted C3-C12cycloalkyl, optionally substituted C3-C12cycloalkenyl, optionally substituted C2-C12 heterocycloalkyl, C2-C12 heterocycloalkenyl, optionally substituted C6-C18aryl, optionally substituted C1-C18heteroaryl, and acyl, or any two or more of Ra, Rb, Rc and Rd, when taken together with the atoms to which they are attached form a heterocyclic ring system with 3 to 12 ring atoms.

In some embodiments each optional substituent is independently selected from the group consisting of: halogen, ═O, ═S, —CN, —NO2, —CF3, —OCF3, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyi, arylamino, sulfonyl, alkylsulfonyl, arylsulfonyl, aminosulfonyl, aminoalkyi, —COOH, —SH, and acyl.

Examples of particularly suitable optional substituents include F, Cl, Br, I, CH3, CH2CH3, OH, OCH3, CF3, OCF3, NO2, NH2, and CN.

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Acyl” means an R—C(═O)— group in which the R group may be an alkyl, cycloalkyl, heterocycloalkyl, aryl or heteroaryl group as defined herein. Examples of acyl include acetyl and benzoyl. The group may be a terminal group or a bridging group. If the group is a terminal group it is bonded to the remainder of the molecule through the carbonyl carbon.

“Acylamino” means an R—C(═O)—NH— group in which the R group may be an alkyl, cycloalkyl, heterocycloalkyl, aryl or heteroaryl group as defined herein. The group may be a terminal group or a bridging group. If the group is a terminal group it is bonded to the remainder of the molecule through the nitrogen atom.

“Alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. The alkenyl group is preferably a 1-alkenyl group. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₁-C₆ alkenyloxy groups. The group may be a terminal group or a bridging group. If the group is a terminal group it is bonded to the remainder of the molecule through the oxygen atom.

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified.

“Mono-alkylamino” means an Alkyl-NH— group, in which alkyl is as defined herein.

“Dialkylamino” means a (alkyl)₂N— group, in which each alkyl may be the same or different and are each as defined herein for alkyl. The alkyl group is preferably a C₁-C₆alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which alkyl is as defined herein, x is 1 or 2, and the sum of X+Y=2. The group may be a terminal group or a bridging group. If the group is a terminal group it is bonded to the remainder of the molecule through the carbonyl carbon.

“Alkyloxy” refers to an alkyl-O— group in which alkyl is as defined herein. Preferably the alkyloxy is a C₁-C₆alkyloxy. Examples include, but are not limited to, methoxy and ethoxy. The group may be a terminal group or a bridging group.

“Alkyloxyalkyl” refers to an alkyloxy-alkyl- group in which the alkyloxy and alkyl moieties are as defined herein. The group may be a terminal group or a bridging group. If the group is a terminal group it is bonded to the remainder of the molecule through the alkyl group.

“Alkyloxyaryl” refers to an alkyloxy-aryl- group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₆ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl- group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroaryl” refers to an alkyloxy-heteroaryl- group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl- group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S—(═O)— group in which alkyl is as defined herein. The alkyl group is preferably a C₁-C₆ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₆alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₁-C₆alkynyloxy groups. The group may be a terminal group or a bridging group. If the group is a terminal group it is bonded to the remainder of the molecule through the oxygen atom.

“Aminoalkyl” means an NH₂-alkyl- group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇cycloalkenyl group are fused together to form a cyclic structure, such as tetrahydronaphthyl, indenyl or indanyl. The group may be a terminal group or a bridging group. Typically an aryl group is a C₆-C₁₈ aryl group.

“Arylalkenyl” means an aryl-alkenyl- group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl- group in which the aryl and alkyl moieties are as defined herein. Preferred arylalkyl groups contain a C₁₋₅alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH—, in which aryl is as defined herein. di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl- group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₆-C₁₈aryloxy, more preferably a C₆-C₁₀aryloxy. The group may be a terminal group or a bridging group. If the group is a terminal group it is bonded to the remainder of the molecule through the oxygen atom.

“Arylsulfonyl” means an aryl-S(═O)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. A cycloalkenyl group typically is a C₃-C₁₂alkenyl group. The group may be a terminal group or a bridging group.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group may be a terminal group or a bridging group.

“Cycloalkylalkyl” means a cycloalkyl-alkyl- group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl- group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl- group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₁-C₆cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₁-C₆cycloalkenyloxy. The group may be a terminal group or a bridging group. If the group is a terminal group it is bonded to the remainder of the molecule through the oxygen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight- or branched-chain alkyl group preferably having from 2 to 12 carbons, more preferably 2 to 6 carbons in the chain, in which one or more of the carbon atoms (and any associated hydrogen atoms) are each independently replaced by a heteroatomic group selected from S, O, P and NR′ where R′ is selected from the group consisting of H, optionally substituted C₁-C₁₂ alkyl, optionally substituted C₃-C₁₂ cycloalkyl, optionally substituted C₆-C₁₈ aryl, and optionally substituted C₁-C₁₈ heteroaryl. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heteroalkyloxy” refers to a heteroalkyl-O— group in which heteroalkyl is as defined herein. Preferably the heteroalkyloxy is a C₂-C₆heteroalkyloxy. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. A heteroaryl group is typically a C₁-C₁₈heteroaryl group. The group may be a terminal group or a bridging group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridylmethyl. The group may be a terminal group or a bridging group. If the group is a terminal group it is bonded to the remainder of the molecule through the alkyl group.

“Heteroarylalkenyl” means a heteroaryl-alkenyl- group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl- group in which the heteroaryl and heteroalkyl moieties are as defined herein. The group may be a terminal group or a bridging group. If the group is a terminal group it is bonded to the remainder of the molecule through the heteroalkyl group.

“Heteroaryloxy” refers to a heteroaryl-O— group in which the heteroaryl is as defined herein. Preferably the heteroaryloxy is a C₁-C₁₈heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl group as defined herein but containing at least one double bond. A heterocycloalkenyl group typically is a C₂-C₁₂heterocycloalkenyl group. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₂-C₁₂heterocycloalkyl group. The group may be a terminal group or a bridging group.

“Heterocycloalkylalkyl” refers to a heterocycloalkyl-alkyl- group in which the heterocycloalkyl and alkyl moieties are as defined herein. Exemplary heterocycloalkylalkyl groups include (2-tetrahydrofuryl)methyl, (2-tetrahydrothiofuranyl) methyl. The group may be a terminal group or a bridging group. If the group is a terminal group it is bonded to the remainder of the molecule through the alkyl group.

“Heterocycloalkylalkenyl” refers to a heterocycloalkyl-alkenyl- group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl- group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the Heterocycloalkenyloxy is a C₁-C₆Heterocycloalkenyloxy. The group may be a terminal group or a bridging group. If the group is a terminal group it is bonded to the remainder of the molecule through the oxygen atom.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH)ₓ. In groups of this type n is typically from 1 to 10, more preferably from 1 to 6, most preferably 1 to 3. x is typically 1 to 6, more preferably 1 to 3.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art. For those compounds where there is the possibility of geometric isomerism the applicant has drawn the isomer that the compound is thought to be although it will be appreciated that the other isomer may be the correct structural assignment.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propanoic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

“Prodrug” means a compound that undergoes conversion to a compound of formula (I) within a biological system, usually by metabolic means (e.g. by hydrolysis, reduction or oxidation). For example an ester prodrug of a compound of formula (I) containing a hydroxyl group may be convertible by hydrolysis in vivo to the parent molecule. Suitable esters of compounds of formula (I) containing a hydroxyl group, are for example acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates. As another example an ester prodrug of a compound of formula (I) containing a carboxy group may be convertible by hydrolysis in vivo to the parent molecule. (Examples of ester prodrugs are those described by F. J. Leinweber, Drug Metab. Res., 18:379, 1987). Similarly, an acyl prodrug of a compound of formula (I) containing an amino group may be convertible by hydrolysis in vivo to the parent molecule (Many examples of prodrugs for these and other functional groups, including amines, are described in Prodrugs: Challenges and Rewards (Parts 1 and 2); Ed V. Stella, R. Borchardt, M. Hageman, R. Oliyai, H. Maag and J Tilley; Springer, 2007).

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

Specific compounds of the invention include the following:
Ethyl 3,5-diphenyl-1H-pyrrole-2-carboxylate
Ethyl 5-(4-methoxyphenyl)-3-m-tolyl-1H-pyrrole-2-carboxylate
Ethyl 3-(furan-2-yl)-5-(4-methoxyphenyl)-1H-pyrrole-2-carboxylate
Ethyl 3-(3-nitrophenyl)-5-p-tolyl-1H-pyrrole-2-carboxylate
Ethyl 3-(4-cyanophenyl)-5-p-tolyl-1H-pyrrole-2-carboxylate
Ethyl 5-(4-chlorophenyl)-3-(pyridin-3-yl)-1H-pyrrole-2-carboxylate
Ethyl 5-(4-chlorophenyl)-3-(naphthalen-2-yl)-1H-pyrrole-2-carboxylate
Ethyl 3,5-di(thiophen-2-yl)-1H-pyrrole-2-carboxylate
Ethyl 3-(4-bromophenyl)-5-(thiophen-2-yl)-1H-pyrrole-2-carboxylate
Ethyl 5-(3,4-dimethoxyphenyl)-3-(4-(trifluoromethyl)phenyl)-1H-pyrrole-2-carboxylate
Methyl 5-(3-methoxyphenyl)-3-(4-(trifluoromethyl)phenyl)-1H-pyrrole-2-carboxylate
Ethyl 5-(3,4-dimethoxyphenyl)-3-(pyridin-4-yl)-1H-pyrrole-2-carboxylate
Methyl 3-(3-nitrophenyl)-5-p-tolyl-1H-pyrrole-2-carboxylate
Ethyl 3-(thiophen-2-yl)-5-(3,4,5-trimethoxyphenyl)-1H-pyrrole-2-carboxylate
Ethyl 5-(3-methoxyphenyl)-3-(pyridin-4-yl)-1H-pyrrole-2-carboxylate
or a geometric isomer or a pharmaceutically acceptable salt or prodrug thereof.

The compounds have the ability to inhibit topoisomerase activity. Such ability may be a result of the compounds acting directly and solely on topoisomerase to modulate/potentiate biological activity. However, it is understood that the compounds may also act at least partially on other factors associated with topoisomerase activity.

The inhibition of topoisomerase activity may be carried out in any of a number of well-known ways in the art. In circumstances where it is desired to inhibit topoisomerase in a mammal, the inhibition of topoisomerase typically involves administering the compound to a mammal containing the topoisomearase activity.

Accordingly the compounds may find a multiple number of applications in which their ability to inhibit topoisomerase activity of the type mentioned above can be utilised.

Accordingly compounds of the invention would be expected to have useful therapeutic properties especially in relation to cancer treatment, wherein said cancer is cancer of the lung, breast, colon, prostate, melanoma, pancreas, stomach, liver, brain, kidney, uterus, cervix, ovaries, urinary tract, gastral intestinal, other tumors which grown in an anatomical site other than the bloodstream, blood born tumors, colon, rectal, or combinations thereof.

Administration of compounds within Formula (I) can be by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose. In various embodiments the activator compound may be selectively toxic or more toxic to rapidly proliferating cells, e.g. cancerous tumours, than to normal cells.

In using the compounds of the invention they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. As such in some embodiments the present invention provides a pharmaceutical composition including a compound of Formula (I) and a pharmaceutically acceptable carrier, diluent or excipient. The compositions are prepared in manners well known in the art.

The invention in other embodiments provides a pharmaceutical pack or kit comprising one or more containers filled with one or more of the ingredients of the pharmaceutical compositions of the invention. In such a pack or kit can be found a container having a unit dosage of the agent(s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

The compounds of the invention may be used or administered in combination with one or more additional drug(s) for the treatment of the disorder/diseases mentioned. The components can be administered in the same formulation or in separate formulations. If administered in separate formulations the compounds of the invention may be administered sequentially or simultaneously with the other drug(s).

In addition to being able to be administered in combination with one or more additional drugs, the compounds of the invention may be used in a combination therapy. When this is done the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylatedisostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, patches, sprays, ointments and inhalants. The active compound is mixed under sterile conditions with a pharmaceutically acceptable carrier and any needed preservatives, buffers, or propellants which may be required.

The amount of compound administered will preferably treat and reduce or alleviate the condition. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

### SYNTHESIS OF COMPOUNDS OF THE INVENTION

The agents of the various embodiments may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3^{rd} Edition, John Wiley & Sons, 1991. Alternatively, other reactions disclosed herein or known in the art will be recognized as having applicability for preparing other compounds of the various embodiments.

Reagents useful for synthesizing compounds may be obtained or prepared according to techniques known in the art.

The symbols, abbreviations and conventions in the processes, schemes, and examples are consistent with those used in the contemporary scientific literature. Specifically but not meant as limiting, the following abbreviations may be used in the examples and throughout the specification.
g (grams)
L (liters)
Hz (Hertz)
mol (moles)
RT (room temperature)
min (minutes)
MeOH (methanol)
CHCl₃ (chloroform)
DCM (dichloromethane)
DMSO (dimethylsulfoxide)
EtOAc (ethyl acetate)
mg (milligrams)
mL (milliliters)
psi (pounds per square inch)
mM (millimolar)
μM (micromolar)
ng (nanogram)
° C. (degree celsius)
NMR (nuclear magnetic resonance)
ppm (parts per million)
ESI (electro spray ionization)
HRMS (high-resolution-mass spectrometry)
HPLC (high-performance liquid chromatography)
MHz (megahertz)
h (hours)
TLC (thin layer chromatography)
EtOH (ethanol)
CDCl₃ (deuterated chloroform)
HCl (hydrochloric acid)
DMF (N, N-dimethylformamide)
THF (tetrahydrofuran)
K₂CO₃ (potassium carbonate)
Na₂SO₄ (sodium sulfate)
RM (reaction mixture)
IC50 (inhibitory concentration resulting in 50% decreased activity)
PI (propidiumiodide)
FITC (fluorescein isothiocyanate)
MTT (3-(4, 5-dimethylthiazol-2-yl)-2,5-diphenyltetrazolium bromide)
JC-1 (5, 5′, 6, 6′-tetrachloro-1, 1′, 3, 3′-tetraethylbenzimidazolylcarbocyanine iodide)
PBS (phosphate buffer saline)
EDTA (ethylenediaminetetraacetic acid)
SDS (sodium dodecyl sulfate)
DTT (dithiothreitol)
BSA (bovine serum albumin)
Topo I (topoisomerase I)
Topo IIα (topoisomerase II alpha)
Etoposide (4′-demethyl-epipodophyllotoxin 9-[4,6-O—(R)-ethylidene-beta-D-glucopyranoside])
DNA (deoxyribonucleic acid)

Unless otherwise indicated, all temperatures are expressed in ° C. (degree celsius). All reactions conducted at room temperature unless otherwise mentioned.

All the solvents and reagents used are commercially available and purchased from Sigma Aldrich, Fluka, Acros, Spectrochem, Alfa Aesar, Avra, Qualigens, Merck, Rankem and Leonid Chemicals.

¹H NMR spectra were recorded on a Bruker AV 300. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), m (multiplet), or br (broad).

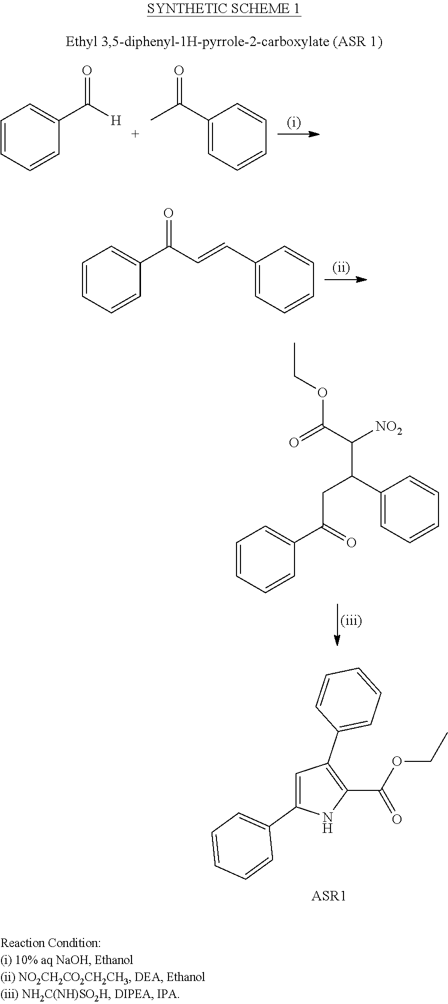

Off white solid (78%): mp 140-1420 C (lit 139-1400 C); Rf 0.5 (2:8 EtOAc: Hexane); IR (KBr, Cm-1) 3311, 2978, 1661, 1603, 1462, 1437, 1365, 1268, 1134, 819, 764, 657, 505; 1H NMR (400 MHz, CDCl3) δ 9.42 (s, 1H, NH), 7.60-7.59 (m, 4H, ArH), 7.43-7.38 (m, 4H, ArH), 7.34-7.32 (m, 2H, ArH), 6.629-6.622 (d, J=2.8 Hz, 1H, C4H), 4.28-4.26 (q, J=2.0 Hz, 2H, OCH2CH3), 1.25-1.23 (t, J=7.2 Hz, 3H, OCH2CH3); 13C NMR (100 MHz, CDCl3) δ 161.2, 135.3, 135.0, 133.4, 131.0, 129.5, 129.0, 127.9, 127.6, 127.0, 124.7, 118.5, 109.9, 60.3, 14.1; HRMS (ESI) m/z Calcd for C₁₉H₁₇NO₂ [M+Na]+ 314.3438, found 314.3439. HPLC: >95%.

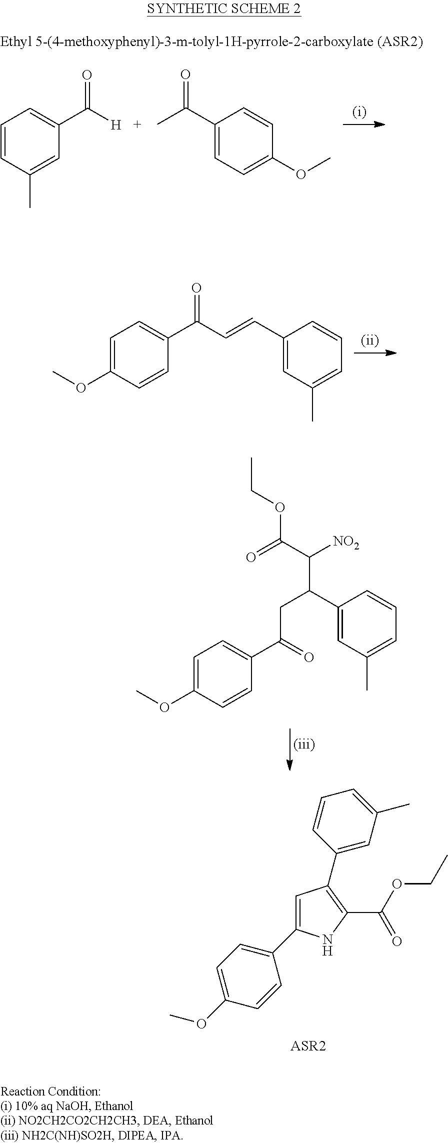

White solid (76%): mp 130-1320 C; Rf 0.6 (3:7 EtOAc: Hexane); IR (KBr, Cm-1) 3314, 2999, 2833, 1664, 1476, 1448, 1297, 1266, 1242, 1016, 802, 719, 530; 1H NMR (400 MHz, CDCl3) δ 9.24 (s, 1H, NH), 7.54-7.51 (m, 2H, ArH), 7.41-7.39 (d, J=9.6 Hz, 2H, ArH), 7.29-7.26 (t, J=7.8 Hz, 1H, ArH), 7.148-7.144 (m, 1H, ArH), 6.98-6.95 (m, 2H, ArH), 6.52-6.652 (d, J=3.2 Hz, 1H, C4H), 4.29-4.26 (q, J=3.6 Hz, 2H, OCH2CH3), 3.86 (s, 3H, OCH3), 2.39 (s, 3H, CH3), 1.29-1.26 (t, J=7.6 Hz, 3H, OCH2CH3); 13C NMR (100 MHz, CDCl3) δ 161.4, 151.9, 137.4, 135.7, 134.7, 133.9, 130.2, 127.7, 127.5, 126.6, 126.3, 126.0, 114.4, 109.0, 60.2, 55.3, 21.4, 14.1; HRMS (ESI) m/z Calcd for C₂₁H₂₁NO₃ [M+Na]+ 358.3963, found 358.3965; HPLC: >96%.

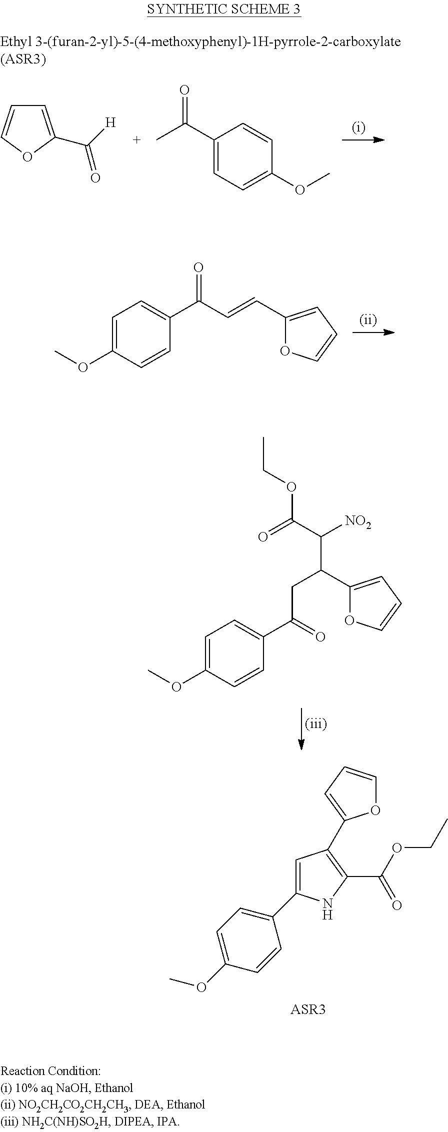

Pale pink solid (70%): mp 134-1360 C; Rf 0.65 (2:8 EtOAc: Hexane); IR (KBr, Cm-1) 3339, 2984, 2839, 1667, 1483, 1467, 1271, 1237, 1186, 1026, 828, 747, 523; 1H NMR (400 MHz, CDCl3) δ 9.07 (s, 1H, NH), 7.54-7.52 (m, 2H, ArH), 7.455-7.450 (m, 1H, ArH), 7.22-7.21 (d, J=2.8 Hz, 1H, ArH), 6.97-6.95 (d, J=7.2 Hz, 2H, ArH), 6.86-6.85 (d, J=2.5 Hz, 1H, C4H), 6.49-6.48 (m, 1H, ArH), 4.43-4.38 (q, J=5.8 Hz, 2H, OCH2CH3), 3.85 (s, 3H, OCH3), 1.44-1.41 (t, J=5.7 Hz, 3H, OCH2CH3); 13C NMR (100 MHz, CDCl3) δ 160.5, 159.5, 148.8, 141.2, 135.5, 126.1, 123.6, 122.9, 116.6, 114.4, 111.5, 109.4, 106.1, 60.5, 55.3, 14.5; HRMS (ESI) m/z Calcd for C₁₈H₁₇NO₄ [M+Na]+ 334.3319, found 334.3321; HPLC: >95%.

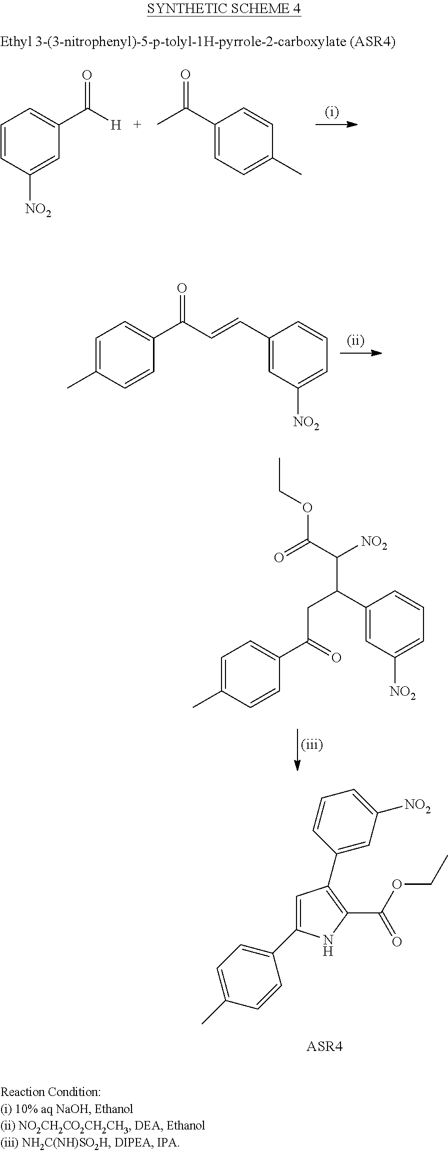

Yellow solid (71%): mp 148-1500 C; Rf 0.65 (2:8 EtOAc: Hexane); IR (KBr, Cm-1) 3327, 2923, 2853, 1536, 1345, 1268, 1205, 1096, 1024, 883, 869, 685, 508; 1H NMR (400 MHz, CDCl3) δ 9.37 (s, 1H, NH), 8.50-8.49 (t, J=1.6 Hz, 1H, ArH), 8.18-8.16 (m, 1H, ArH), 7.94-7.92 (m, 1H, ArH), 7.55-7.49 (m, 3H, ArH), 7.26-7.25 (m, 2H, ArH), 6.629-6.623 (d, J=2.3 Hz, 1H, C4H), 4.31-4.27 (q, J=5.8 Hz, 2H, OCH2CH3), 2.39 (s, 3H, CH3), 1.27-1.24 (t, J=5.8 Hz, 3H, OCH2CH3); 13C NMR (100 MHz, CDCl3) δ 161.0, 147.8, 138.3, 136.8, 136.6, 130.8, 129.8, 128.5, 128.4, 127.7, 127.6, 124.7, 124.5, 118.2, 109.2, 60.7, 21.2, 14.1; HRMS (ESI) m/z Calcd for C₂₀H₁₈N₂O₄ [M+Na]+ 373.3679, found 373.3683; HPLC: >94%.

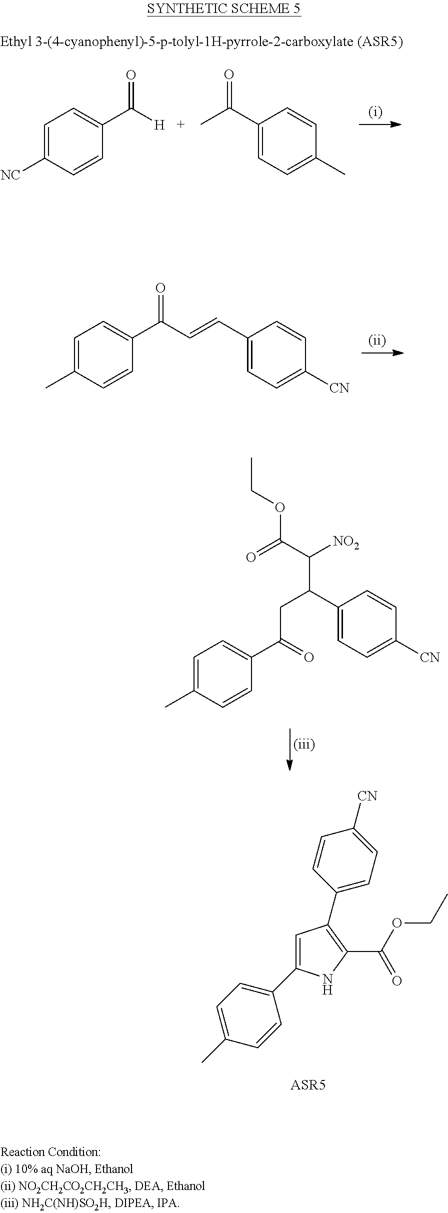

White solid (69%): mp 196-1980 C; Rf 0.65 (2:8 EtOAc: Hexane); IR (KBr, Cm-1) 3311, 3110, 2924, 2854, 2227, 1661, 1503, 1476, 1445, 1289, 1207, 1135, 1112, 1081, 729, 560; 1H NMR (400 MHz, CDCl3) δ 9.34 (s, 1H, NH), 7.71-7.69 (m, 2H, ArH), 7.67-7.65 (m, 2H, ArH), 7.49-7.47 (d, J=6.6 Hz, 2H, ArH), 7.26-7.24 (d, J=7.2 Hz, 2H, ArH), 6.587-6.581 (d, J=2.3 Hz, 1H, C4H), 4.30-4.27 (q, J=5.6 Hz, 2H, OCH2CH3), 2.39 (s, 3H, CH3), 1.29-1.25 (t, J=5.2 Hz, 3H, OCH2CH3); 13C NMR (100 MHz, CDCl3) δ 160.7, 140.0, 138.3, 136.0, 131.4, 131.3, 130.1, 129.8, 127.7, 124.7, 119.2, 118.5, 110.4, 109.2, 60.6, 21.2, 14.2; HRMS (ESI) m/z Calcd for C₂₁H₁₈N₂O₂ [M+Na]+ 353.3798, found 353.3799; HPLC: >96%.

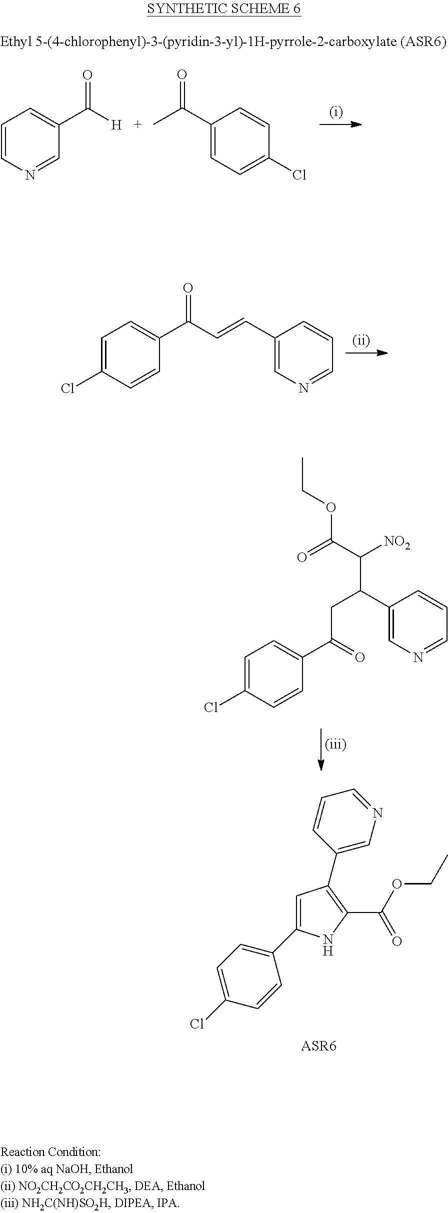

White solid (52%); mp 156-1580 C; Rf 0.4 (1:1 EtOAc: Hexane); IR (KBr, Cm-1) 3325, 2978, 2926, 1896, 1672, 1572, 1482, 1380, 1206, 1091, 1027, 808, 649, 511, 466; 1H NMR (400 MHz, CDCl3) δ 9.41 (s, 1H, NH), 8.787-8.782 (m, 1H, ArH), 8.56-8.55 (d, J=5.0 Hz, 1H, ArH), 7.92-7.89 (m, 1H, ArH), 7.54-7.52 (d, J=6.6 Hz, 2H, ArH), 7.42-7.41 (d, J=6.8 Hz, 2H, ArH), 7.32-7.30 (m, 1H, ArH), 6.61-6.60 (d, J=2.4 Hz, 1H, C4H), 4.30-4.26 (q, J=5.6 Hz, 2H, OCH2CH3), 1.26-1.24 (t, J=5.6 Hz, 3H, OCH2CH3); 13C NMR (100 MHz, CDCl3) δ 160.8, 150.0, 148.1, 136.7, 134.6, 133.9, 130.9, 129.4, 129.3, 126.0, 122.5, 119.4, 109.9, 60.7, 14.1; HRMS (ESI) m/z Calcd for C₁₈H₁₅ClN₂O₂ [M+Na]+ 349.7769, found 349.7773; HPLC: >98%.

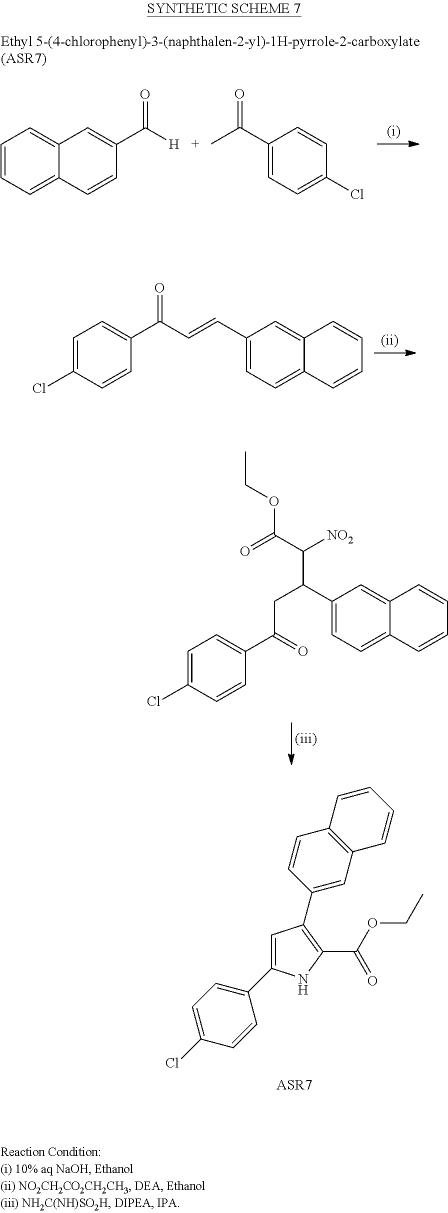

White solid (75%): mp 142-1440 C; Rf 0.7 (2:8 EtOAc: Hexane); IR (KBr, Cm-1) 3320, 2976, 2926, 1673, 1480, 1384, 1283, 1139, 1028, 900, 647, 504, 477; 1H NMR (400 MHz, CDCl3) δ 9.34 (s, 1H, NH), 8.049-8.045 (m, 1H, ArH), 7.86-7.83 (m, 3H, ArH), 7.73-7.70 (d, J=8.2 Hz, 1H, ArH), 7.56-7.54 (m, 2H, ArH), 7.49-7.47 (m, 2H, ArH), 7.42-7.40 (m, 2H, ArH), 6.71-6.70 (d, J=2.5 Hz, 1H, C4H), 4.30-4.26 (q, J=5.7 Hz, 2H, OCH2CH3), 1.25-1.22 (t, J=5.6 Hz, 3H, OCH2CH3); 13C NMR (100 MHz, CDCl3) δ 161.2, 133.7, 133.3, 132.5, 132.3, 129.5, 129.2, 128.1, 128.07, 128.00, 127.5, 126.9, 125.9, 125.8, 119.1, 110.4, 60.5, 14.2; HRMS (ESI) m/z Calcd for C₂₃H₁₈ClNO₂ [M+Na]+ 398.8475, found 398.8479; HPLC: ≥95%.

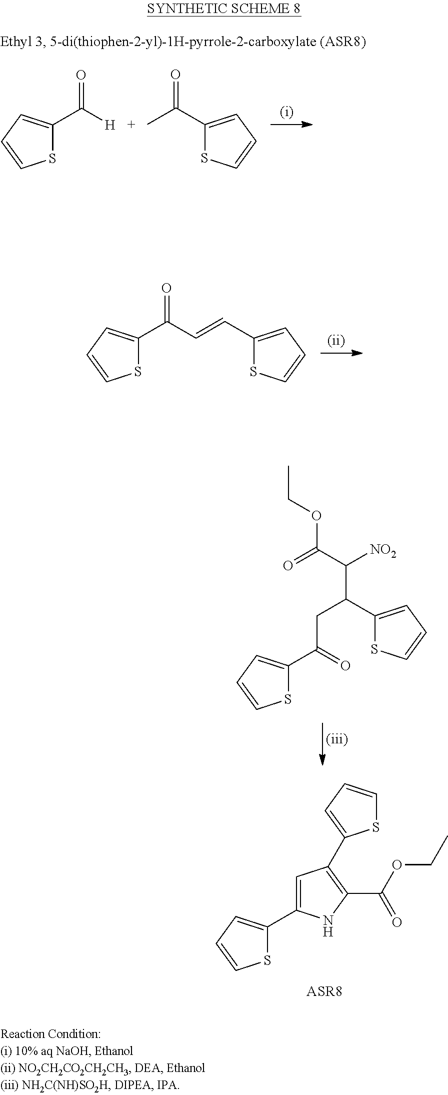

Off white solid (68%): mp 104-1060 C; Rf 0.6 (3:7 EtOAc: Hexane); IR (KBr, Cm-1) 3302, 3103, 2993, 2934, 1665, 1590, 1514, 1456, 1276, 1166, 1078, 939, 502, 474; 1H NMR (400 MHz, CDCl3) δ 9.07 (s, 1H, NH), 7.57-7.56 (m, 1H, ArH), 7.30-7.28 (m, 2H, ArH), 7.24-7.23 (m, 1H, ArH), 7.09-7.05 (m, 2H, ArH), 6.63-6.62 (d, J=2.3 Hz, 1H, C4H), 4.40-4.35 (q, J=5.6 Hz, 2H, OCH2CH3), 1.39-1.36 (t, J=5.6 Hz, 3H, OCH2CH3); 13C NMR (100 MHz, CDCl3) δ 160.5, 136.0, 133.6, 129.9, 127.9, 127.1, 127.0, 125.6, 125.1, 124.9, 123.4, 117.6, 109.9, 60.7, 14.4; HRMS (ESI) m/z Calcd for C₁₅H₁₃NO₂S₂ [M+Na]+ 326.3992, found 326.3997; HPLC: ≥95%.

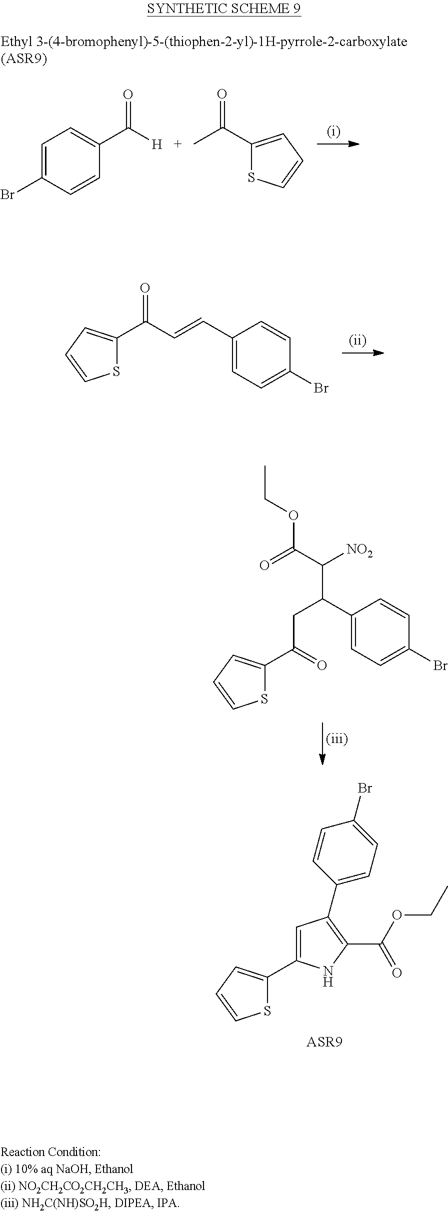

Off white solid (66%): mp 174-1760 C; Rf 0.55 (3:7 EtOAc: Hexane); IR (KBr, Cm-1) 3315, 3100, 2976, 2926, 1712, 1659, 1530, 1478, 1409, 1320, 1199, 1023, 829, 706, 645, 482; 1H NMR (400 MHz, CDCl3) δ 9.16 (s, 1H, NH), 7.50-7.44 (m, 4H, ArH), 7.29-7.28 (m, 1H, ArH), 7.23-7.22 (m, 1H, ArH), 7.08-7.07 (m, 1H, ArH), 6.486-6.480 (d, J=2.3 Hz, 1H, C4H), 4.30-4.26 (q, J=5.6 Hz, 2H, OCH2CH3), 1.26-1.29 (t, J=5.7 Hz, 3H, OCH2CH3); 13C NMR (100 MHz, CDCl3) δ 160.8, 133.8, 133.6, 132.0, 131.1, 130.7, 130.0, 127.9, 124.9, 123.3, 121.2, 118.1, 110.0, 60.5, 14.2; HRMS (ESI) m/z Calcd for C₁₇H₁₄BrNO₂S [M+Na]+ 399.2676, found 399.2679 [M+2+Na]+; HPLC: ≥95%.

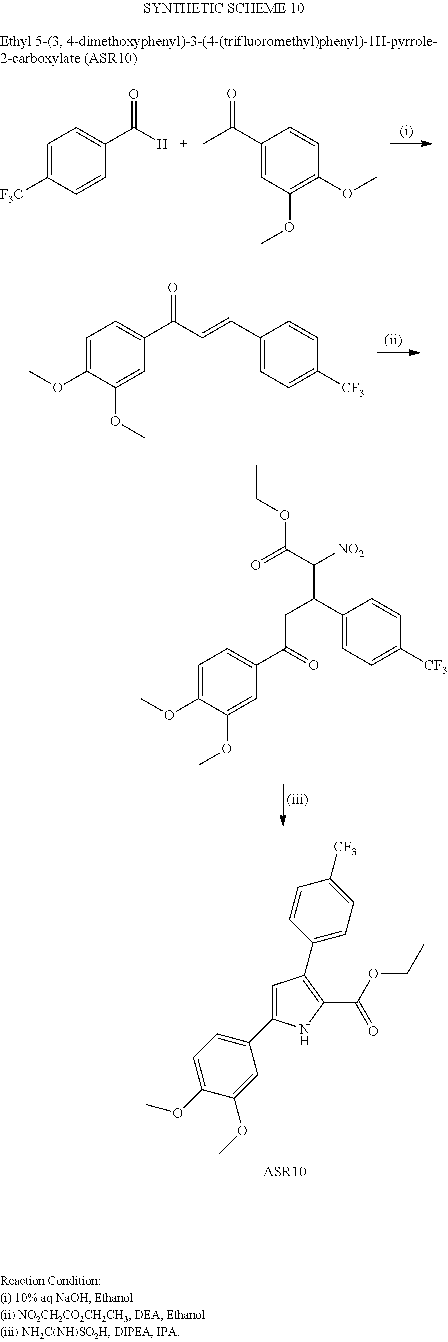

Pale yellow solid (80%): mp 120-1220 C; Rf 0.65 (3:7 EtOAc: Hexane); IR (KBr, Cm-1) 3325, 2980, 2839, 1671, 1617, 1461, 1294, 1152, 1078, 845, 730, 634, 534; 1H NMR (400 MHz, CDCl3) δ 9.27 (s, 1H, NH), 7.71-7.69 (d, J=6.8 Hz, 2H, ArH), 7.65-7.62 (m, 2H, ArH), 7.16-7.14 (m, 1H, ArH), 7.074-7.070 (m, 1H, ArH), 6.94-6.92 (d, J=6.6 Hz, 1H, ArH), 6.538-6.532 (d, J=2.3 Hz, 1H, C4H), 4.30-4.26 (q, J=5.6 Hz, 2H, OCH2CH3), 3.96 (s, 3H, OCH3), 3.93 (s, 3H, OCH3), 1.27-1.24 (t, J=5.6 Hz, 3H, OCH2CH3); 13C NMR (100 MHz, CDCl3) δ 160.7, 149.0, 132.4, 132.0, 128.0, 127.4, 127.1, 125.2, 125.19, 125.15, 125.13, 125.11, 120.5, 111.0, 110.7, 109.4, 109.2, 61.5, 55.99, 55.95, 14.0; HRMS (ESI) m/z Calcd for C₂₂H₂₀F₃NO₄ [M+Na]+ 442.3937, found 442.3935; HPLC: ≥96%.

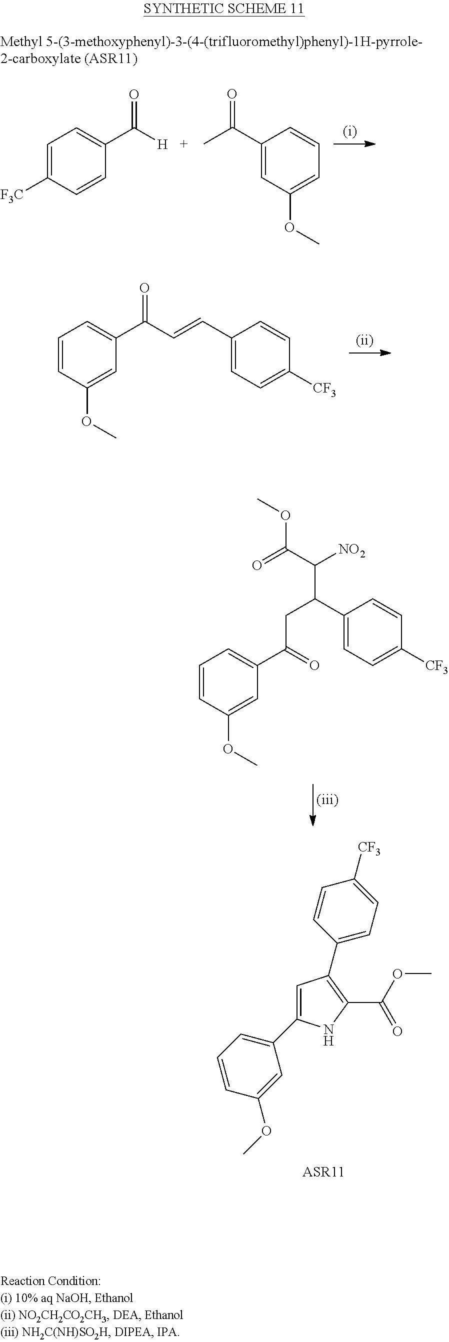

White solid (60%); mp 122-1240 C; Rf 0.65 (3:7 EtOAc: Hexane); IR (KBr, Cm-1) 3332, 2980, 2843, 1670, 1621, 1461, 1297, 1156, 1078, 845, 737, 634, 539; 1H NMR (400 MHz, CDCl3) δ 9.29 (s, 1H, NH), 7.71-7.69 (d, J=6.4 Hz, 2H, ArH), 7.64-7.63 (d, J=6.4 Hz, 2H, ArH), 7.16-7.14 (d, J=6.6 Hz, 2H, ArH), 7.07 (s, 1H, ArH), 6.94-6.92 (d, J=6.6 Hz, 1H, ArH), 6.54-6.53 (d, J=2.2 Hz, 1H, C4H), 3.96 (s, 3H, OCH3), 3.93 (s, 3H, OCH3); 13C NMR (100 MHz, CDCl3) δ 160.7, 149.0, 132.4, 132.0, 128.0, 127.4, 127.1, 125.2, 125.19, 125.15, 125.13, 125.11, 121.5, 120.5, 111.0, 110.7, 109.4, 109.2, 55.9, 51.5; HRMS (ESI) m/z Calcd for C₂₀H₁₆F₃NO₃ [M+Na]+ 398.3411, found 398.3418; HPLC: >96%.

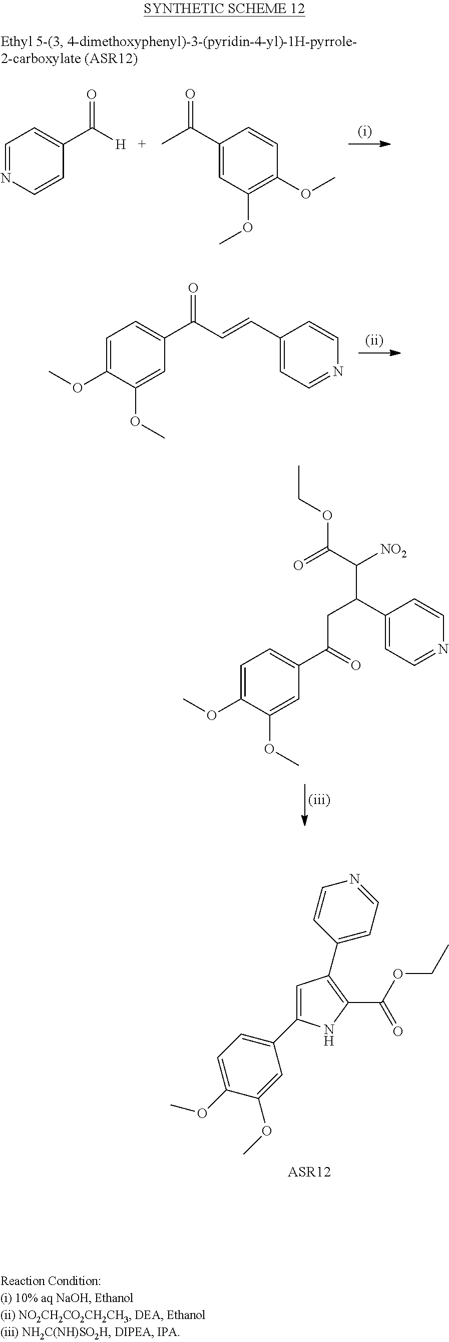

Yellow solid (54%): mp 136-1380 C; Rf 0.5 (1:1 EtOAc: Hexane); IR (KBr, Cm-1) 3442, 3060, 2934, 2852, 2721, 1707, 1605, 1509, 1440, 1294, 1235, 1112, 832, 803, 766, 670, 466; 1H NMR (400 MHz, CDCl3) δ 9.49 (s, 1H, NH), 8.62 (s, 2H, ArH), 7.56-7.54 (m, 2H, ArH), 7.18-7.16 (t, J=5.2 & 2.8 Hz, 1H, ArH), 7.09 (s, 1H, ArH), 6.95-6.92 (m, 1H, ArH), 6.58-6.57 (d, J=3.2 Hz, 1H, C4H), 4.32-4.29 (q, J=3.2 Hz, 2H, OCH2CH3), 3.969 (s, 3H, OCH3), 3.960 (s, 3H, OCH3), 1.29-1.27 (t, J=3.6 Hz, 3H, OCH2CH3); 13C NMR (100 MHz, CDCl3) δ 160.8, 149.4, 149.0, 148.9, 136.1, 124.2, 124.1, 123.7, 118.6, 117.5, 111.6, 108.9, 108.3, 56.0, 55.9, 14.1; HRMS (ESI) m/z Calcd for C₂₀H₂₀N₂O₄ [M+Na]+ 375.3835, found 375.3837; HPLC: >96%.

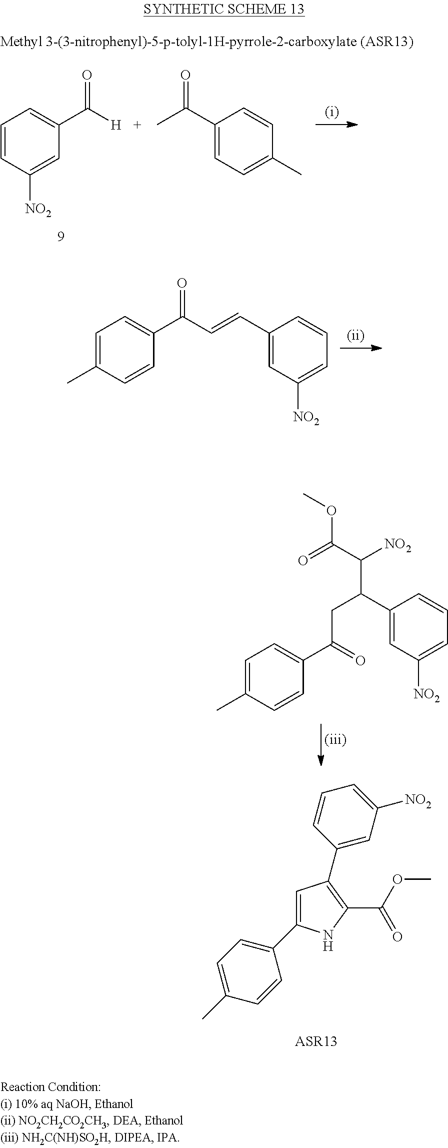

Yellow solid (71%): mp 152-1540 C; Rf 0.65 (2:8 EtOAc: Hexane); IR (KBr, Cm-1) 3330, 2923, 2853, 1536, 1355, 1268, 1205, 1096, 1024, 887, 869, 685, 508; 1H NMR (400 MHz, CDCl3) δ 9.37 (s, 1H, NH), 8.50-8.49 (t, J=1.6 Hz, 1H, ArH), 8.18-8.16 (m, 1H, ArH), 7.94-7.92 (m, 1H, ArH), 7.55-7.49 (m, 3H, ArH), 7.26-7.25 (m, 2H, ArH), 6.629-6.623 (d, J=2.3 Hz, 1H, C4H), 3.87 (s, 3H, OCH3), 2.39 (s, 3H, CH3); 13C NMR (100 MHz, CDCl3) δ 161.0, 147.8, 138.3, 136.8, 136.6, 130.8, 129.8, 128.5, 128.4, 127.7, 127.6, 124.7, 124.5, 118.2, 109.2, 51.7, 21.2; HRMS (ESI) m/z Calcd for C₁₉H₁₆N₂O₄ [M+Na]+ 359.3413, found 359.3418; HPLC: >95%.

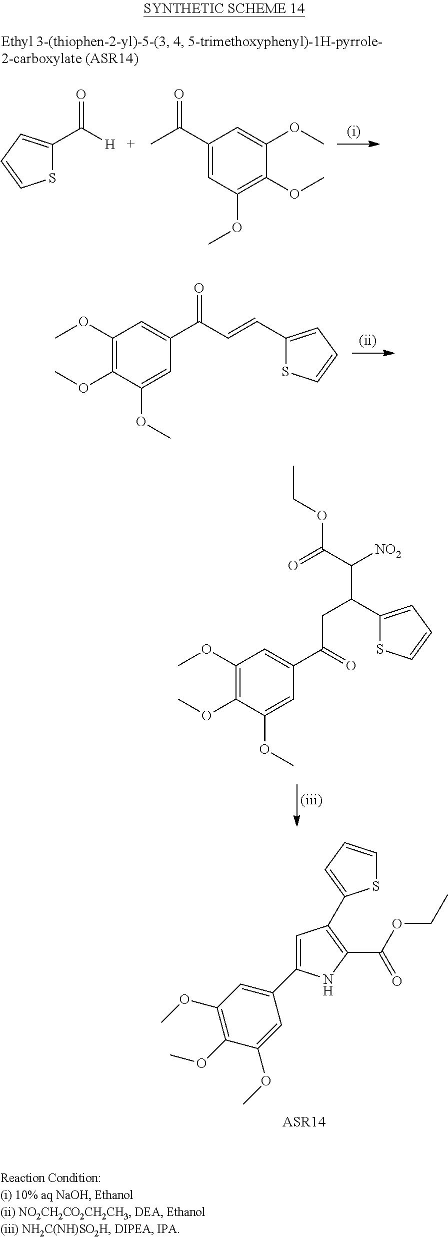

Pale brown solid (62%): mp 100-1020 C; Rf 0.6 (3:7 EtOAc: Hexane); IR (KBr, Cm-1) 3315, 3100, 2926, 1712, 1659, 1530, 1478, 1450, 1320, 1283, 1199, 1023, 829, 706, 522; 1H NMR (400 MHz, CDCl3) δ 9.18 (s, 1H, NH), 7.56-7.55 (m, 1H, ArH), 7.31-7.29 (m, 1H, ArH), 7.08-7.06 (m, 1H, ArH), 6.76 (s, 2H, ArH), 6.65-6.64 (d, J=2.5 Hz, 1H, C4H), 4.41-4.36 (q, J=5.8 Hz, 2H, OCH2CH3), 3.94 (s, 6H, (OCH3)2), 3.88 (s, 3H, OCH3), 1.40-1.37 (t, J=5.6 Hz, 3H, OCH2CH3); 13C NMR (100 MHz, CDCl3) δ 161.4, 152.4, 151.9, 137.5, 135.7, 134.7, 133.8, 130.0, 127.7, 127.5, 126.6, 126.3, 114.3, 109.2, 60.0, 55.3, 54.4, 14.1; HRMS (ESI) m/z Calcd for C₂₀H₂₁NO₅S [M+Na]+ 410.4494, found 410.4497; HPLC: >96%.

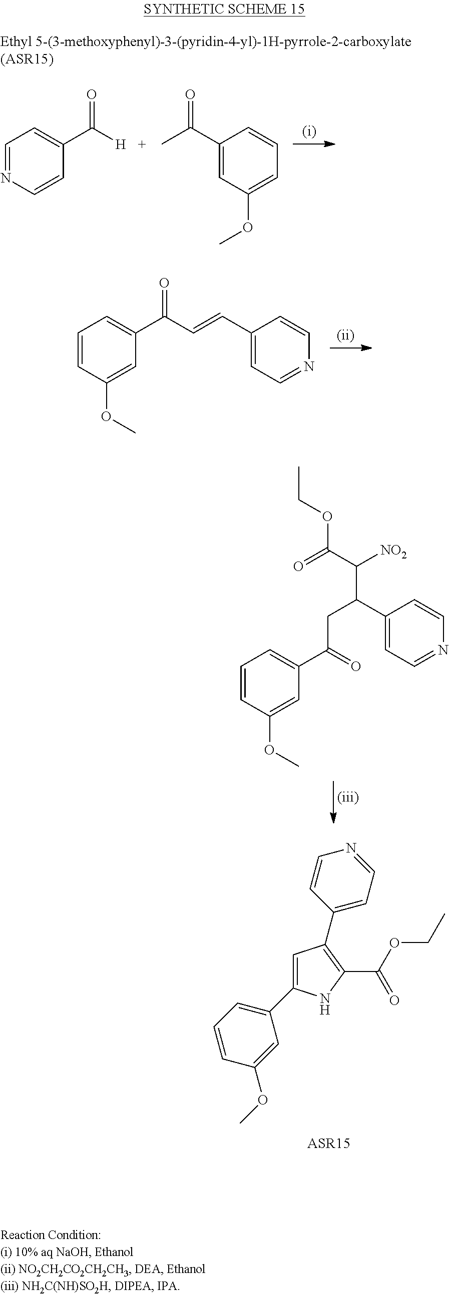

Yellow solid (56%): mp 134-1360 C; Rf 0.55 (1:1 EtOAc: Hexane); IR (KBr, Cm-1) 3442, 3065, 2934, 2852, 2724, 1707, 1605, 1509, 1441, 1294, 1235, 1112, 832, 806, 766, 670, 468; 1H NMR (400 MHz, CDCl3) δ 9.49 (s, 1H, NH), 8.62 (s, 2H, ArH), 7.56-7.54 (m, 2H, ArH), 7.18-7.16 (t, J=5.2 & 2.8 Hz, 1H, ArH), 7.09 (s, 1H, ArH), 6.95-6.92 (m, 2H, ArH), 6.58-6.57 (d, J=3.2 Hz, 1H, C4H), 4.32-4.29 (q, J=3.2 Hz, 2H, OCH2CH3), 3.96 (s, 3H, OCH3), 1.29-1.27 (t, J=3.6 Hz, 3H, OCH2CH3); 13C NMR (100 MHz, CDCl3) δ 160.8, 149.4, 149.0, 148.9, 136.1, 124.2, 124.1, 123.7, 118.6, 117.5, 111.6, 108.9, 108.3, 60.7, 55.9, 14.1; HRMS (ESI) m/z Calcd for C₂₀H₂₀N₂O₄ [M+Na]+ 345.3578, found 345.3582; HPLC: >96%.

### Biological Activity

Chemicals & reagents: All the chemicals used in present study were purchased from Sigma-Aldrich, TopoGEN. Inc, USA and SRL, India.

Cell Lines and Culture

Human cancer cell lines, K562, MCF7, HeLa and HEK 293T were purchased from National Centre for Cell Science, Pune, India. Cells were grown in RPMI 1640/MEM/DMEM supplemented with 10% heat-inactivated fetal bovine serum (FBS), 100 U/mL of Penicillin, and 100 μg of streptomycin/mL and incubated at 37° C. in a humidified atmosphere containing 5% CO2.

Nalm6, Molt4 and Reh cells were provided by Dr. Sathees C. Raghavan, Department of Biochemistry, IISc, Bangalore. The cells were grown under the similar culture conditions as mentioned above.

The human T cell leukemia cell line Molt4, Human acute lymphoblastic leukemia (ALL) cell line Nalm6, Human acute myelocytic leukemia (AML) cell line Reh, chronic myelogenous leukemia (CML) cell line K562, Human breast adenocarcinoma cell lines like MCF7 and human kidney cell line HEK 293T (Human embryonic kidney cell line) were selected for the purpose of preliminary anticancer screening of newly synthesized compounds (ASR1 to ASR15). To check the cytotoxicity, trypan blue dye exclusion assays, MTT assay were employed as described earlier Further, Cell cycle analysis, Mitochondrial membrane potential, Annexin-V FITC staining assay were also performed in order to understand the mode of cell death after treatment with ASR6. Further, ASR6 was also tested for its ability to inhibit topoisomerase I and II activity in vitro.

Cytotoxicity Assays

MTT Assay

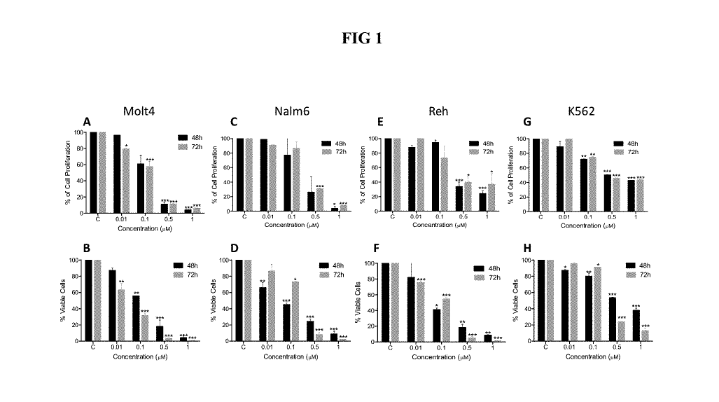

Cytotoxic effect of the newly synthesized compounds against leukemic cells was assessed using 3-(4, 5-dimethyl-2-yl02, 5-diphenyl tetrazolium bromide (MTT) assay. After exposure of compound at different concentration (0.01, 0.1, 0.5, 1, 10, 50 and 100 μM) cells were harvested and transferred into 96 well plate, MTT (5 mg/ml) was added and incubate at 37° C. for 4 h and formazan crystals were dissolved by adding solubilizing agent (10% SDS and 50% dimethyl formamide) for 1 h, finally plate was read at 570 nm (Bio-Rad, USA). Results were represented as inhibition of percentage of cell proliferation in bar diagram (n=3) (FIGS. 1 A, C, E and G).

Trypan Blue Dye Exclusion Assay

To determine the growth inhibitory activity of the drug, 0.5×10⁵ cell/ml were plated in 24 well (Biofil, USA.) in 1 ml of complete medium and treated with various concentration of the compound to test, cells were stained with 0.4% trypan blue and counted on a hemocytometer after treatment with compounds at 48 and 72 h time point. Results were represented as number of viable cells/ml in bar diagram (FIGS. 1 B, D, F, and H) (n=3).

TABLE 1
Antiproliferation studies of 2, 3, 5 trisubstituted pyrrole derivatives

| Comp. | Cytotoxicity IC 50 in μM (Cell lines) | | | | | | |
|---|---|---|---|---|---|---|---|
| Name | Molt4 | Nalm6 | Reh | K562 | MCF7 | HeLa | HEK293T |
| ASR1 | >50 | >50 | >100 | >100 | >100 | >100 | N.T |
| ASR2 | >50 | >50 | >100 | >100 | >100 | >100 | N.T |
| ASR3 | >50 | >50 | >100 | >100 | >100 | >100 | N.T |
| ASR4 | >50 | >50 | >100 | >100 | >100 | >100 | N.T |
| ASR5 | >50 | >50 | >100 | >100 | >100 | >100 | N.T |
| ASR6 | 0.195 | 0.344 | 0.360 | 0.512 | 0.884 | 0.749 | 0.866 |
| ASR7 | 30.77 | >50 | >100 | >100 | >100 | >100 | N.T |
| ASR8 | 35.26 | >50 | >100 | >100 | >100 | >100 | N.T |
| ASR9 | 50 | >50 | >100 | >100 | >100 | >100 | N.T |
| ASR10 | 7.96 | 19.96 | >100 | >100 | >100 | >100 | N.T |
| ASR11 | 16.97 | 9.53 | >100 | >100 | >100 | >100 | N.T |
| ASR12 | 26.10 | 14.26 | >100 | >100 | >100 | >100 | N.T |
| ASR13 | 8.84 | 10.99 | >100 | >100 | >100 | >100 | N.T |
| ASR14 | 15.19 | 10.48 | >100 | >100 | >100 | >100 | N.T |
| ASR15 | 12.93 | 5.4 | >100 | >100 | >100 | >100 | N.T |

Cell Cycle Analysis

The DNA content analysis was performed by using Flow cytometer, Molt4 cells seeded (0.5×10⁵ cells/ml) in 6 well plate and treated with different concentrations of ASR6 (0.01, 0.1, 0.25 and 0.5 μM) at 37° C. for 48 h. After incubation cells were harvested, processed and fixed in 80% chilled ethanol. Overnight treated with RNase-A (50 μg/ml) incubated at 37° C. Further cells stained with propidium iodide (5 μg/ml) and cell cycle progression was monitored using flow cytometer (FACSVerse™ BD Biosciences, USA). A minimum of 10000 cells were recorded. Results were analyzed using Flowing Software (Version 2.5) and plotted histograms.

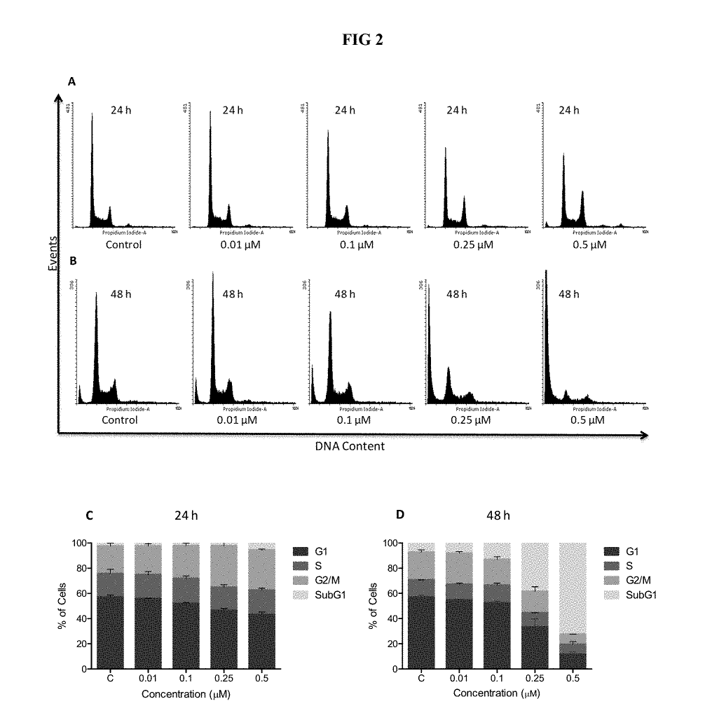

Results revealed that ASR6 inhibited cell cycle progression of G2/M arrest (cell cycle arrest) at 24 h and SubG1 phase (apoptosis) at 48 h in Molt4 cells in a concentration dependent manner (FIG. 2A, B). In FIG. 2 A, B histograms representing different phases of cell cycle distribution of ASR6 (0.01, 0.1, 0.25 and 0.5 μM for 24 h and 48 h) treated Molt4 cells is shown. Interestingly the treatment of ASR6 in Molt4 cells was shown prominent cell cycle arrest at G2/M phase at 24 h time point. FIG. 2 C, D shows a bar diagram (n=3) representing percentage of cell population in various phases (G1, S, G2/M and SubG1) of the cell cycle in vehicle control and ASR6 treated Molt4 cells.

Mitochondrial Membrane Potential Assay

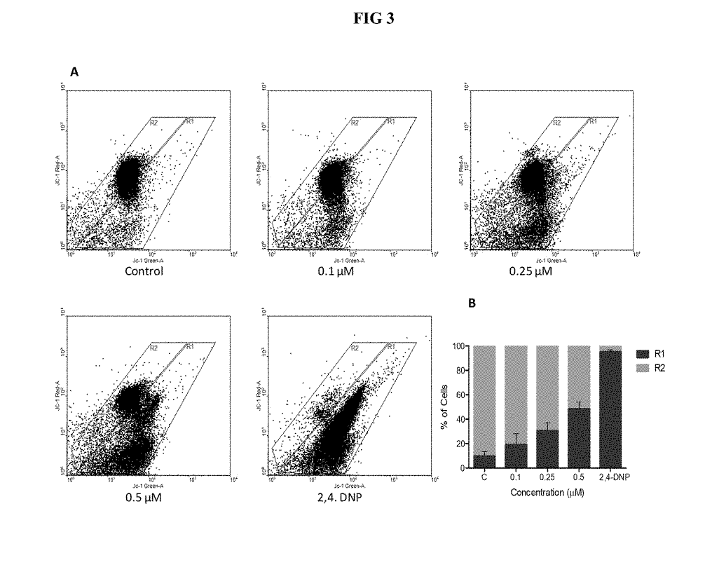

Molt4 cells were seeded in 12 well plate (0.5×10⁵ cells/ml), treated with different concentration of ASR6 for 48 h, (DMSO treated cells was used as vehicle control), washed with 1× phosphate buffered saline and stained with JC-1 dye (5,5′,6,6′-Tetrachloro-1,1′,3,3′-tetraethylbenzimidazolo carbocyanine iodide (0.5 μg/ml) (Calbiochem, USA) in 1 ml of media, incubated at 37° C. for 30 min. Samples were washed with phosphate buffered saline and finally resuspended the cell pellet in 300 μl of 1× phosphate buffered saline and acquired (FACSVerse™, BD Biosciences, USA) using Cell Quest Pro Software. Minimum of 10,000 cells were acquired per sample and 2, 4-Dinitrophenol (2, 4-DNP) used as positive control. Results were analyzed Flowing Software (Version 2.5) and data were presented. Result showed reduction in mitochondrial membrane potential in ASR6 treated Molt4 cells (FIG. 3). FIG. 3 A shows a Dot plot representing JC-1 stained Molt4 cells at various concentrations of ASR6 (0.1, 0.25 and 0.5 μM) for 48 h, 2, 4-DNP treated Molt4 cells. FIG. 3 B shows a bar graph (n=2) showing percentage of red versus green fluorescence cells which indicates high and low MMP, respectively.

Annexin V-FITC Staining for Apoptosis Studies

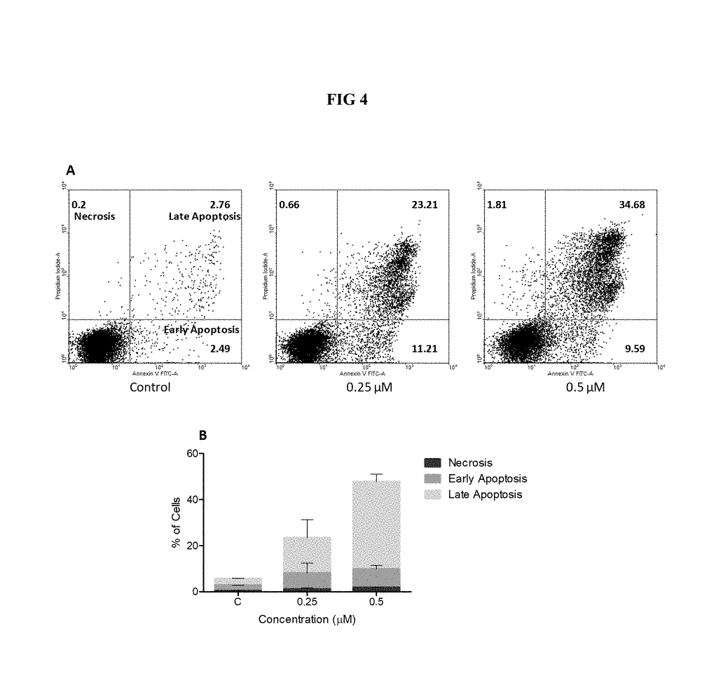

To identify induction of apoptosis after treatment withASR6 in Molt4 cells, annexin V-FITC and propidium iodide staining was carried out. Briefly, Molt4 cells (0.5×10⁵ cells per ml) were seeded in 24 well plate and treated with ASR6 (0.25 & 0.5 μM; 48 h). Cells were washed with 1× phosphate buffered saline and suspended in cell binding buffer, stained with Annexin V-FITC (0.2 mg/ml) and propidium iodide (0.05 mg/ml)(Santacruz, USA), and incubated in dark for 20 min at room temperature. Finally cells were subjected to FACS analysis (FACSVerse™BD Bioscience, USA) (10,000 cells were acquired). The results were analyzed in WinMDI 2.9 Software, bar diagram was plotted using at least two independent experiments with error bar. The result showed significant increase in apoptotic cell population of ASR6 treated Molt4 cells (FIG. 4). FIG. 4 A shows the effect of treatment with ASR6 on Molt4 cells (0.25 & 0.5 μM) for 48 h and processed for annexin V-FITC/PI double staining. In lower left quadrant shows cells which are negative for both annexin V-FITC and PI, lower right shows only annexin V positive cells which are in early stage of apoptosis, upper left shows only PI positive cells which are necrotic/dead cells and upper right shows both annexin V and PI positive cells, which are undergoing late apoptosis. FIG. 4 B shows a bar graph (n=2) shows the percentage of cell population in different stages of apoptosis.

Topoisomerase Inhibition Studies

Topoisomerase I Assay

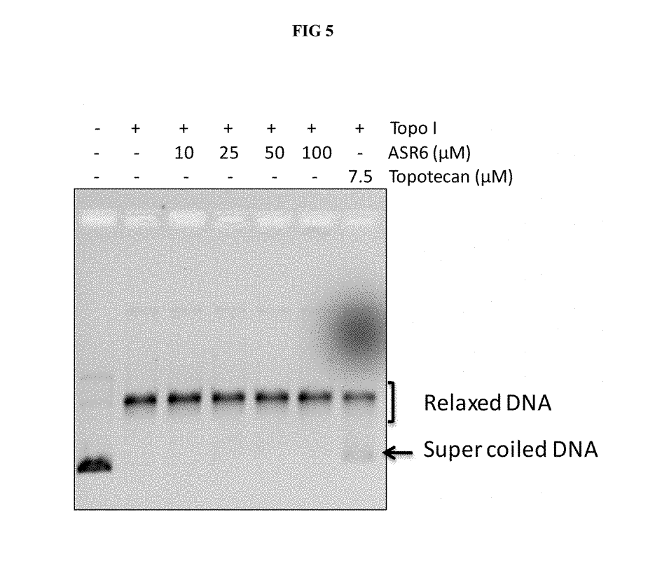

In order to determine the ability of ASR6 of topoisomerase I activity assay was performed, in a reaction mixture containing supercoiled plasmid DNA (pBS-SK+) was isolated using Gen Elute plasmid miniprep kit (Sigma Aldrich), according to manual instructions. ˜200 ng plasmid and two units of recombinant human DNA topoisomerase I (TopoGEN. Inc) along with the different concentrations of ASR6 (0, 10, 25, 50 and 100 μM) was added at RT for 5 min; equal amount of DMSO was used as vehicle control. Reaction was carried out at 37° C. for 30 min in a relaxation buffer 1× topoI buffer (50 mM Tris-HCl (pH 8.0), 10 mM NaCl, 10 mM MgCl2, 5 mM ATP, 0.5 mM dithiothreitol and 30 μg BSA/ml) and reactions were terminated by adding 5× stop buffer containing 5% sarkosyl, 0.0025% bromophenol blue, 25% glycerol. The DNA samples were electrophoresed on 1% agarose gel at 45 volts for 4 h with 0.5×TBE (Tris-borate-EDTA). The gels were stained for 30 min in milli Q water containing ethidium bromide (0.5 mg/ml) followed by distaining for 30 min in milli Q water and images were taken (FIG. 5). Results revealed that ASR6 does not inhibit topoisomerase I activity in dose dependent manner. Topotecan (Topo I inhibitor) was used as positive control for topoisomerase I.

Topoisomerase IIα Assay

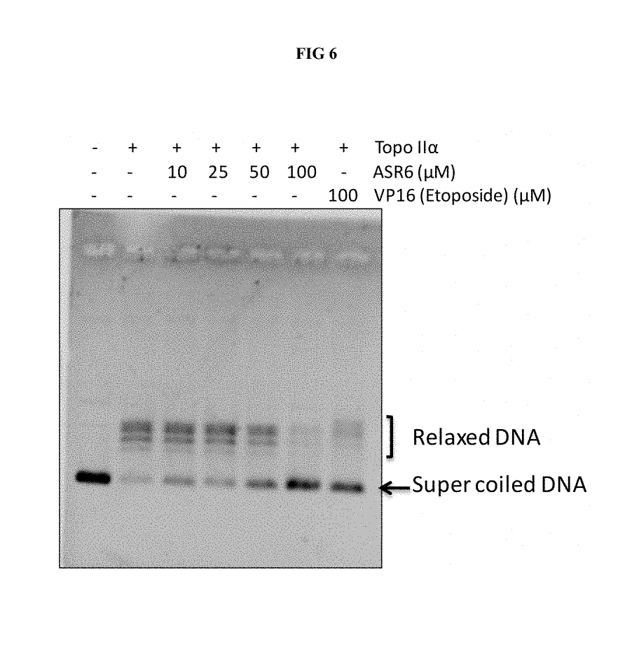

The topoisomerase IIα assay was performed, in a reaction mixture containing supercoiled plasmid DNA (pBS-SK+) was isolated using Gen Elute plasmid miniprep kit (Sigma Aldrich), according to manual instructions. ˜200 ng plasmid and two units of recombinant human DNA topoisomerase IIα (TopoGEN. Inc) along with the different concentrations of ASR6 (0, 10, 25, 50 and 100 μM) was added at RT for 5 min; equal amount of DMSO was used as vehicle control. Reaction was carried out at 37° C. for 30 min in a relaxation buffer 1× topo II buffer (50 mM Tris-HCl (pH 8.0), 10 mM NaCl, 10 mM MgCl2, 5 mM ATP, 0.5 mM dithiothreitol and 30 μg BSA/ml) and reactions were terminated by adding 5× stop buffer containing 5% sarkosyl, 0.0025% bromophenol blue, 25% glycerol. The DNA samples were electrophoresed on 1% agarose gel at 45 volts for 4 h with 0.5×TBE (Tris-borate-EDTA). The gels were stained for 30 min in milli Q water containing ethidium bromide (0.5 mg/ml) followed by distaining for 30 min in milli Q water and images were taken. Results revealed that in vitro topoisomerase assays using topoisomerase I and Ha were showed that ASR6 can inhibit topoisomerase Ha enzymatic activity to relax the supercoiled DNA significantly as compared to the topoisomerase I in dose dependent manner. VP16 (Topo II inhibitor) were used as positive control (FIG. 6).

Statistical Analysis

The results were analyzed using graph pad prism, values were expressed as mean±SEM for samples and statistical analysis was performed. One-way ANOVA followed by Dunnett test, in each case experimental samples were compared with control and significance was determined. The values were considered as statistically significant, if the p-value was equal to or less than 0.05. (0.05*, 0.005**, 0.0005***).

1. A compound of formula (I): 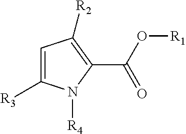
wherein:
R1 is selected from a group consisting of —CH3 and —CH2CH3;
R2 is selected from a group consisting of optionally substituted C6-C18 aryl and optionally substituted C1-C18 heteroaryl;
R3 and R4 are independently selected from a group consisting of H, halogen, CN, —NO2, SH, CF3, OH, CO2H, CONH2, OCF3, optionally substituted C1-C12alkyl, optionally substituted C1-C12 haloalkyl, optionally substituted C2-C12 alkenyl, optionally substituted C2-C12 alkynyl, optionally substituted C1-C12alkyloxy, optionally substituted C1-C12haloalkyloxy, optionally substituted C2-C12 heteroalkyl, optionally substituted C3-C12cycloalkyl, optionally substituted C3-C12 cycloalkenyl, optionally substituted C2-C12 heterocycloalkyl, optionally substituted C2-C12 heterocycloalkenyl, optionally substituted C6-C18 aryl, and optionally substituted C1-C18heteroaryl;
or a pharmaceutically acceptable salt or N-oxide thereof.

2. (canceled)

3. (canceled)

4. (canceled)

5. A compound according to claim 1 wherein R3 is selected from a group consisting of an optionally C6-C18 aryl, and optionally substituted C1-C18heteroaryl.

6. A compound according to claim 1 wherein R4 is H.

7. A compound according to claim 1 wherein each optional substituent is independently selected from the group consisting of H, CH3, CH2CH3, CH2CH2CH3, CH(CH3)2, (CH2)3CH3, Cl, Br, F, I, OH, NO2, NH2, CN, OCH3, OCH2CH3, CF3, and OCF3.

8. A compound according to claim 1 selected from the group consisting of:
Ethyl 3,5-diphenyl-1H-pyrrole-2-carboxylate;
Ethyl 5-(4-methoxyphenyl)-3-m-tolyl-1H-pyrrole-2-carboxylate;
Ethyl 3-(furan-2-yl)-5-(4-methoxyphenyl)-1H-pyrrole-2-carboxylate;
Ethyl 3-(3-nitrophenyl)-5-p-tolyl-1H-pyrrole-2-carboxylate;
Ethyl 3-(4-cyanophenyl)-5-p-tolyl-1H-pyrrole-2-carboxylate;
Ethyl 5-(4-chlorophenyl)-3-(pyridin-3-yl)-1H-pyrrole-2-carboxylate;
Ethyl 5-(4-chlorophenyl)-3-(naphthalen-2-yl)-1H-pyrrole-2-carboxylate;
Ethyl 3,5-di(thiophen-2-yl)-1H-pyrrole-2-carboxylate;
Ethyl 3-(4-bromophenyl)-5-(thiophen-2-yl)-1H-pyrrole-2-carboxylate;
Ethyl 5-(3,4-dimethoxyphenyl)-3-(4-(trifluoromethyl)phenyl)-1H-pyrrole-2-carboxylate;
Methyl 5-(3-methoxyphenyl)-3-(4-(trifluoromethyl)phenyl)-1H-pyrrole-2-carboxylate;
Ethyl 5-(3,4-dimethoxyphenyl)-3-(pyridin-4-yl)-1H-pyrrole-2-carboxylate;
Methyl 3-(3-nitrophenyl)-5-p-tolyl-1H-pyrrole-2-carboxylate;
Ethyl 3-(thiophen-2-yl)-5-(3,4,5-trimethoxyphenyl)-1H-pyrrole-2-carboxylate;
Ethyl 5-(3-methoxyphenyl)-3-(pyridin-4-yl)-1H-pyrrole-2-carboxylate.
or pharmaceutically acceptable salt, or prodrug thereof.

9. A pharmaceutical composition including a compound according to claim 1 and a pharmaceutically acceptable diluent, excipient or carrier.

10. A method for treatment of a condition in a mammal the method comprising administering an effective amount of a compound according to claim 1, wherein said condition can be treated by inhibition of topoisomerase-II activity.

11. (canceled)

12. (canceled)

13. A method according to claim 10 wherein the condition is selected from the group consisting of Cancers such as premalignant and malignant hyperproliferative diseases such as cancers of the breast, pancreas, skin, prostate, cervix, uterus, colon, bladder, esophagus, stomach, lung, larynx, Liver, oral cavity, pancreas, blood and lymphatic system, metaplasias, dysplasias, neoplasias, leukoplakias and papillomas of the mucous membranes and in the treatment of Kaposis sarcoma, Cutaneous T Cell Lymphoma, promyelocyticleukemia, Non-Small-Cell Lung Cancer, Kidney Cancer (Advanced Renal Cell Cancer), Gastrointestinal Cancer, Mesothelioma and Bronchial Metaplasia;

14. Use of a compound according to claim 1 in the preparation of a medicament.

15. (canceled)

16. A use according to claim 14 wherein the medicament is for the treatment of a condition can be prevented or treated by inhibition of topoisomerase-II activity

17. A use according to claim 16 wherein the condition is selected from the group consisting of Cancers such as premalignant and malignant hyperproliferative diseases such as cancers of the breast, pancreas, skin, prostate, cervix, uterus, colon, bladder, esophagus, stomach, lung, larynx, Liver, oral cavity, pancreas, blood and lymphatic system, metaplasias, dysplasias, neoplasias, leukoplakias and papillomas of the mucous membranes and in the treatment of Kaposis sarcoma, Cutaneous T Cell Lymphoma, promyelocyticleukemia, Non-Small-Cell Lung Cancer, Kidney Cancer (Advanced Renal Cell Cancer), Gastrointestinal Cancer, Mesothelioma and Bronchial Metaplasia.

18. (canceled)
